(19) United States
(12) Patent Application Publication
(10) Pub. No.: US 2023/0372687 A1
(43) Pub. Date: Nov. 23, 2023

(54) CAPSULE DEVICE

(71) Applicant: Novo Nordisk A/S, Bagsvaerd (DK)

(72) Inventors: Aghiad Ghazal, Koebenhavn SV (DK); Drago Sticker, Malmoe (SE); Jacob Pyung Hwa Jepsen, Frederiksberg (DK); Cody Edward Cleveland, Koebenhavn K (DK); Jorrit Jeroen Water, Frederiksberg (DK); Brian Mouridsen, Fredensborg (DK)

(21) Appl. No.: 18/030,586

(22) PCT Filed: Oct. 8, 2021
(86) PCT No.: PCT/EP2021/077969

§ 371 Date: Apr. 6, 2023

(30) Foreign Application Priority Data
Oct. 9, 2020 (EP) 20201193.8

(51) Int. Cl. A61M 31/00

(52) U.S. Cl. CPC A61M 31/002; A61M 2205/8225

(57) ABSTRACT

A capsule device (100) suitable for insertion or ingestion into a lumen, such as a gastrointestinal lumen, of a subject. The capsule device (100) comprises:—a capsule housing (110, 130), —a drug outlet (190, 290, 390, 490) arranged relative to the capsule housing (110, 130), —a single capillary duct (125) accommodating a liquid drug substance, —an actuation chamber (A), and—a drug expelling unit, wherein the drug expelling unit is configured for being actuated to expel the liquid drug substance from the single capillary duct through the drug outlet (190), wherein the drug expelling unit comprises a gas expansion unit (150) actuatable to generate pressurized gas in the actuation chamber (A) for exerting load directly onto the liquid drug substance, and wherein a gas release gate (151, 170) is configured for being operated between: c) a first configuration wherein pressurized gas in the actuation chamber (A) is prevented from forcing liquid drug substance from the single capillary duct (125) through the drug outlet (190), and d) a second configuration wherein pressurized gas from the actuation chamber (A) is permitted to force liquid drug substance from the single capillary duct (125) through the drug outlet (190).

The present invention relates to drug delivery devices suitable for ingestion or insertion into a lumen of a human or animal subject, such as a swallowable capsule for delivery of a liquid drug substance to a subject user.

### BACKGROUND OF THE INVENTION

In the disclosure of the present invention reference is mostly made to the treatment of diabetes by delivery of insulin, however, this is only an exemplary use of the present invention.

May people suffer from diseases, such as diabetes, which requires them to receive injections of drugs on a regular and often daily basis. To treat their disease these people are required to perform different tasks which may be considered complicated and may be experienced as uncomfortable. Furthermore, it requires them to bring injection devices, needles and drugs with them when they leave home. It would therefore be considered a significant improvement of the treatment of such diseases if treatment could be based on oral intake of tablets or capsules.

However, such solutions are very difficult to realise, since protein-based drugs will be degraded and digested rather than absorbed when ingested.

To provide a working solution for delivering insulin into the bloodstream through oral intake, the drug has to be delivered firstly into a lumen of the gastrointestinal tract and further into the wall of the gastrointestinal tract (lumen wall). This presents several challenges among which are: (1) The drug has to be protected from degradation or digestion by the acid in the stomach. (2) The drug has to be released while being in the stomach, or in the lower gastrointestinal tract, i.e. after the stomach, which limits the window of opportunity for drug release. (3) The drug has to be delivered at the lumen wall to limit the time exposed to the degrading environment of the fluids in the stomach and in the lower gastrointestinal tract. If not released at the wall, the drug may be degraded during its travel from point of release to the wall or may pass through the lower gastrointestinal tract without being absorbed, unless being protected against the decomposing fluids.

Capsule devices have been proposed for delivery of a drug substance into a lumen or lumen wall. After insertion of the capsule, such as by swallowing the capsule into the GI system of the subject, drug delivery may be performed using an actuator which forces the drug substance 35 from a reservoir through an outlet. Typical capsule devices include a drug reservoir comprising a movable separator, such as a slidable piston arranged between the actuator, such as a compressed spring or a gas expansion unit, and the liquid drug substance in the reservoir.

For such devices it is often a challenge to accommodate a sufficient amount of drug in the capsule device and/or accommodate sufficient energy for effecting a satisfactorily drug deposition at the target for delivery.

Having regard to the above, it is an object of the present invention to provide a capsule device which is improved relative to prior art capsule devices.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in an aspect of the invention, a capsule device for ingestion or insertion into a lumen of a human or animal subject is provided. The capsule device comprises:
a capsule housing,
a drug outlet arranged relative to the capsule housing,
a drug reservoir configured to accommodate a liquid drug substance,
an actuation chamber (A), and
a drug expelling unit, wherein the drug expelling unit is configured for being actuated to expel the liquid drug substance through the drug outlet, wherein the drug expelling unit comprises a gas expansion unit actuatable to generate pressurized gas in the actuation chamber (A), or release pressurized gas from the actuation chamber (A), for exerting load onto the liquid drug substance.

The drug reservoir is provided as a single capillary duct having a first end and a second end, wherein the single capillary duct is configured for fluidically connecting the actuation chamber (A) with the drug outlet, and wherein the liquid drug substance is arranged within the single capillary duct.

A gas release gate is arranged to control flow of pressurized gas from the actuation chamber (A) towards the drug outlet, wherein the gas release gate is configured for being operated between:
a) a first configuration wherein pressurized gas in the actuation chamber (A) is prevented from forcing liquid drug substance from single capillary duct through the drug outlet, and
b) a second configuration wherein pressurized gas from the actuation chamber (A) is permitted to force liquid drug substance from the single capillary duct through the drug outlet.

The gas expansion unit is preferable configured so as to generate pressurized gas in the actuation chamber (A), or release pressurized gas from the actuation chamber (A), for exerting load directly onto the liquid drug substance in the single capillary duct.

Advantages of using a capillary for accommodation and expelling of the liquid drug substance includes the following:
1. Save on space in the device by eliminating the use of piston
2. Reduce the number of moving parts which reduces the complexity of the device
3. Larger drug volume loaded in the device (up to 400 μl)
4. Requires lower energy to drive expelling, for a jet at certain powers (down to 6 bar)
5. Simpler activation method to trigger the device.

In accordance herewith, a particularly simple, and potentially cost-effective solution, is provided.

In certain embodiments of the capsule device, in the second configuration, pressurized gas from the actuation chamber (A) engages directly with the liquid drug substance in the single capillary duct thereby exerting load, i.e., gas pressure, onto the liquid drug substance to force the liquid drug substance towards the drug outlet.

In accordance herewith, for some embodiments, the capsule device does not incorporate a slidable piston, or other movable separation wall, between the actuation chamber (A) and the liquid drug substance.

Typically, in the second configuration during drug expelling, the liquid drug substance in the single capillary duct arranged closest to the actuation chamber (A), and the pressurized gas, define a liquid-gas interface.

In some embodiments, of the capsule device, when in the second configuration, pressurized gas from the actuation chamber (A) engages directly with the liquid drug substance in the single capillary duct to exert load onto the liquid drug substance for moving the liquid drug substance towards the drug outlet.

In some embodiments the liquid drug substance forms a liquid column comprising first and second immiscible liquid substances arranged in series within the single capillary duct, wherein the second liquid substance differs from the first second liquid substance and is arranged upstream from the first liquid substance, and wherein at least the first liquid substance, and optionally the second liquid substance, includes a beneficial agent for providing a therapeutic effect. By forming first and second liquid substances in separate portions within the single capillary duct, the second liquid substance, which may be provided in smaller quantity compared to the first liquid substance, may exhibit different physical and chemical parameters than the first liquid component, and the capsule device may utilize optimized properties for maintaining a proper and well-defined gas/liquid interface between the pressurized gas and the liquid drug substance. This provides more freedom for selecting the first liquid substance which typically will be optimized for a therapeutic effect when administered to a patient.

In further embodiments, the single capillary duct, between the first end and the second end, forms an elongated capillary extending in a non-rectilinear configuration, such as a coiled configuration.

It is to be noted that, although some embodiments according to the invention only includes a single drug outlet with a dedicated drug reservoir in form of a single capillary duct, a dedicated actuation chamber (A), and a dedicated drug expelling unit for that single drug outlet, other embodiments may incorporate multiple such sets of a dedicated drug reservoir in form of a single capillary duct, a dedicated actuation chamber (A), and a dedicated drug expelling unit for each single drug outlet.

It is also to be noted that, for the present invention, the term “capillary duct” is primarily used to convey the information that the single capillary duct forms a narrow, elongated channel wherein a well-defined liquid-gas interface is maintained, i.e., not necessitating the use of moving the liquid inside the channel by capillary action as such.

In some embodiments, the capsule is sized and configured for being ingested into a gastrointestinal lumen.

In some embodiments the capsule device is configured for being inserted or ingested into a lumen wherein the lumen comprises a lumen wall, and wherein the drug outlet comprises a nozzle arrangement configured for needleless liquid je delivery, and wherein the capsule is configured to expel the liquid drug substance through the nozzle arrangement with a penetration velocity allowing the liquid drug substance to penetrate tissue of the lumen wall.

In other embodiments the capsule device is configured for being inserted or ingested into a lumen wherein the lumen comprises a lumen wall, and wherein the drug outlet comprises an injection needle configured to deliver the liquid drug substance from the single capillary duct through a lumen of the injection needle.

The gas expansion unit may in some forms comprise a gas generator configured actuatable to generate pressurized gas in the actuation chamber (A) for exerting load on the liquid drug substance. A burst gate may be arranged between the gas generator and the single capillary duct, the burst gate being configured to release load onto liquid drug substance in the single capillary duct upon increase in gas pressure in the actuation chamber (A) above a threshold pressure level to thereby initiate expelling of the liquid drug substance.

Exemplary embodiments may include a burst gate that comprises a rupturable membrane, such as a burst disc.

In some variants of the capsule device, the capsule device further comprises a trigger arrangement for initiating drug delivery through the drug outlet, e.g. in response to a triggering event. In some forms the trigger arrangement is provided to comprise an environmentally-sensitive mechanism.

In some forms, the capsule device is configured for swallowing by a patient and travelling into a lumen of a GI tract of a patient, such as the small intestine or the large intestine, respectively. The environmentally-sensitive mechanism may in certain embodiments be a GI tract environmentally-sensitive mechanism. The GI tract environmentally-sensitive mechanism may-comprise a trigger member, wherein the trigger member is characterised by at least one of the group comprising:
a) the trigger member comprises a material that degrades, erodes and/or dissolves due to a change in pH in the GI tract;
b) the trigger member comprises a material that degrades, erodes and/or dissolves due to a pH in the GI tract;
c) the trigger member comprises a material that degrades, erodes and/or dissolves due to a presence of an enzyme in the GI tract; and
d) the trigger member comprises a material that degrades, erodes and/or dissolves due to a change in concentration of an enzyme in the GI tract.

In alternative forms, the trigger arrangement may also be or include an electronic trigger

In embodiments wherein the capsule device comprises a gas generator, the gas generator may comprise a trigger arrangement configured to actuate the gas generator.

In further embodiments of the capsule device, the gas expansion unit comprises a pressurized gas canister filled with pressurized gas and comprising a rupturable seal which is so configured, that upon being ruptured, enables pressurized gas from the gas canister to flow to the actuation chamber (A).

In some forms, the gas release gate is defined or comprises said rupturable seal.

The capsule device in forms comprising a pressurized gas canister and a rupturable seal, may further comprises a trigger arrangement comprising a spike, wherein the spike and the pressurized gas canister are arranged for relative movement, and wherein the trigger arrangement comprises means for creating relative movement between the spike and the pressurized gas canister to rupture the rupturable seal.

In still alternative embodiments the capsule device comprises a further version of a gas release gate provided as a release gate associated with the drug outlet for selectively controlling liquid flow through the drug outlet. The release gate may include a trigger arrangement for enabling the release gate to be operated from the first configuration to the second configuration so that pressurized gas in the actuation chamber (A) is allowed to drive out the liquid drug substance from the single capillary duct upon a triggering event. In such embodiments, the release gate may in some forms be provided as a membrane which prior to triggering seals the drug outlet but may become ruptured or otherwise unsealed to enable fluid flow. In capsule devices wherein a release gate is provided at the drug outlet, the gas expansion unit may include pressurized gas so that the liquid drug substance is stored prior to triggering at an elevated pressure level, e.g. at a pressure level equal to the gas pressure level of the pressurized gas.

In some embodiments, the material of the single capillary duct is hydrophobic with contact angle θ towards drug being greater than 40°, such as greater than 60°, such as greater than 80°, and such as greater than 85°. Selecting the material so that the contact angle is much greater than 0°, and preferably close to 90°, will ensure that formation of droplets of the liquid drug substance on the inner surface of the capillary duct is not likely to occur. In some embodiments, the single capillary duct, e.g. the surface material portion of the duct configured for liquid drug contact, is made from a polymeric material.

In some embodiments the cross-sectional shape of the single capillary duct is circular. In other embodiments, the cross-sectional shape of the single capillary duct is generally rectangular or generally square or oval. In still other embodiments, the cross-sectional shape of the single capillary may have a polygonal shape.

In particular embodiments the cross-sectional area of the single capillary duct, at least along a part of its extension, is from 1 mm² to 16 mm², such as from 4 mm² to 10 mm². In some embodiments, the single capillary duct has the same cross-sectional area along a major part of its extension from the first end to the second end, such as along the entire extension from the first end to the second end.

In embodiments wherein the cross-sectional shape of the single capillary duct is circular having an inner diameter between 1 to 5 mm, such as between 2 to 4 mm.

In further embodiments, the capsule housing of the capsule device defines a maximum outer housing dimension (z) prior to administration. In such embodiments, the single capillary duct may be dimensioned having a length measured from the actuation chamber (A) to the drug outlet, wherein said length is at least two times (z), such as at least 5 times (z), such as at least 10 times (z), such as at least 15 times (z), and such as at least 20 times (z). In some forms, the single capillary duct has a length between 5 times (z) and 12 times (z).

In particular embodiments, the single capillary duct has a length between 80 mm and 200 mm, such as between 80 mm and 130 mm, between 130 to r between 150-200 mm.

In some variants of the capsule device, the single capillary duct is shaped to extend along a helical path. In other forms, the single capillary duct is shaped to extend in a meandering configuration inside the capsule housing.

In some forms the single capillary duct is provided in the form of a tubing wherein the tubing may either be manufactured from a rigid material so that it is arranged in a predefined shape. In alternative embodiments, the tubing may be manufactured as a flexible tubing, e.g. wherein the tubing is deformable, such as being able to become coiled after manufacturing of the tubing.

In some embodiments the single capillary duct comprises a total volume of liquid drug substance of 50 to 400 μl, such as between 100 to 300 μl.

In some embodiments, the lumen, such as the small intestine, defines a lumen wall, wherein the drug outlet comprises a jet nozzle arrangement configured for needleless jet delivery. In this way, the ingestible capsule device does not include sharp needle points and a mechanism which actuates and retracts the needle is also not required. By inclusion of the rupturable membrane, such as a burst disc, it is ensured that drug expelling will only commence once sufficient gas pressure acting on the movable separator is present for carrying out a suitable jet injection.

Existing jet injector systems for jet delivery are known in the art. A skilled person would understand how to select an appropriate jet injector that provides the correct jetting power to deliver the therapeutic substance into the lumen wall, for example from WO 2020/106,750 (PROGENITY INC). Further details and examples are provided further on in the application.

For needle-less jet injection embodiments, the capsule may be configured to expel drug substance through the nozzle arrangement with a penetration velocity allowing the drug substance to penetrate tissue of the lumen wall.

In other forms of the capsule, the drug outlet comprises an injection needle wherein the drug substance is expellable through the injection needle.

In exemplary embodiments, the capsule device is configured for swallowing by a patient and travelling into a lumen of a gastrointestinal tract of a patient, such as the stomach, the small intestine or the large intestine, respectively. The capsule of the device may be shaped and sized to allow it to be swallowed by a subject, such as a human.

By the above arrangements an orally administered drug substance can be delivered safely and reliably into the stomach wall or intestinal wall of a living mammal subject.

As used herein, the terms “drug”, “drug substance”, “drug product” or “payload” is meant to encompass any drug formulation capable of being delivered into or onto the specified target site. The drug may be a single drug compound, a premixed or co-formulated multiple drug compound, or even a drug product being mixed by two or more separate drug constituents wherein the mixing is performed either before or during expelling. Representative drugs include pharmaceuticals such as peptides (e.g. insulins, insulin containing drugs, GLP-1 containing drugs as well as derivatives thereof), proteins, and hormones, biologically derived or active agents, hormonal and gene-based agents, nutritional formulas and other substances in both solid, powder or liquid form. Specifically, the drug may be an insulin or a GLP-1 containing drug, this including analogues thereof as well as combinations with one or more other drugs.

### BRIEF DESCRIPTION OF THE DRAWINGS

In the following embodiments of the invention will be described with reference to the drawings, wherein

FIG. 1 is a cross-sectional perspective view of an ingestible capsule 100 according to a first embodiment of the invention, the capsule be,

FIG. 2 is a cross-sectional side view of the ingestible capsule 100 according to the first embodiment of the invention,

FIG. 3 is a perspective view a core member 120 of capsule 100 according to the first embodiment of the invention,

FIG. 4 is a cross-sectional side view of an ingestible capsule 200 according to a second embodiment of the invention

FIG. 5 is a cross-sectional side view of the ingestible capsule 300 according to a third embodiment of the invention,

FIG. 6 is a perspective view a core member 120 of capsule 300 according to the third embodiment of the invention,

FIG. 7 is a cross-sectional side view of the ingestible capsule 400 according to a fourth embodiment of the invention,

FIG. 8 is a perspective view a core member 120 of capsule 400 according to the fourth embodiment of the invention,

FIG. 9 is a graph showing pressure loss for different sized capillaries,

FIGS. 10a and 10b depict schematic representations for a liquid surface for two differently sized capillaries,

FIG. 11 is a graph showing Influence of contact angle and surface tension on max velocity before break-off in capillary,

FIG. 12 is a graph showing the volumetric flow rate Q versus the power for varying nozzle diameters,

FIG. 13 is a graph showing the required capillary diameter for varying nozzle diameters at different power levels, and

FIG. 14 is a graph showing the relationship between power and pressure for varying nozzle diameters.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g., manufactured as a single injection moulded part. The terms “assembly” and “subassembly” do not imply that the described components necessarily can be assembled to provide a unitary or functional assembly or subassembly during a given assembly procedure but is merely used to describe components grouped together as being functionally more closely related.

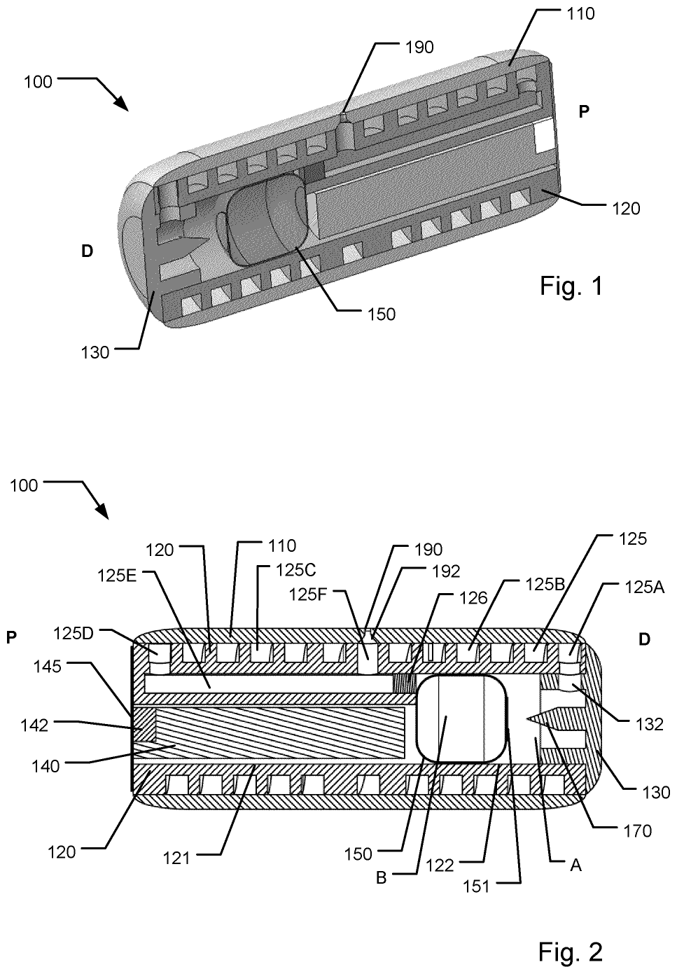

With reference to FIGS. 1, 2 and 3 a first embodiment of a drug delivery device in accordance with the invention will be described, the embodiment being designed to provide a capsule device 100 sized and shaped to be ingested by a patient, or other user, the device being configured for subsequently deploying a triggerable expelling system incorporated in the capsule device, that when triggered in a target lumen of the patient, causes a dose of a liquid drug to be expelled through a drug outlet provided at an external portion of the capsule device 100. It is to be noted that the disclosed ingestible capsule device 100, in the following referred to simply as “capsule”, is only exemplary and, in accordance with the invention, may be provided in other forms having different capsule outer shapes. Also, although the shown outlet provides an outlet nozzle opening for expelling a substance directly through the outlet, the outlet may be provided in alternative forms, such as having an outlet opening associated with an injection needle. The disclosed embodiment relates to a capsule 100 suitable for being ingested by a patient to allow the capsule to enter a lumen of the Gastro-Intestinal tract, more specifically the small intestine, and subsequently to eject a liquid dose of a payload, such as a drug substance at a target location either inside the lumen, or into tissue of the lumen wall surrounding the lumen. In other embodiments, the capsule may be configured for expelling a substance in other locations of the Gastro-Intestinal system, such as the stomach, the large intestine or even in other lumen parts of a subject.

In the shown embodiment capsule 100 the drug substance is intended to be prepared from or provided as a single drug product. Alternatively, the substance may be prepared from at least two drug products. When the substance is prepared by two drug products, a first product may be stored within a first reservoir whereas a second product may be stored in a second drug chamber and mixed prior to expelling or even mixed during expelling through the outlet. In some embodiments, the first drug component is provided initially as a lyophilized drug substance, such as a powder, whereas the second drug component is a reconstitution liquid, such as a diluent. In other embodiments, the two or more drug products are each initially provided as a liquid which are mixed with each other prior to or during drug expelling. However, for simplicity, the following embodiments will disclose variants for expelling a single product only.

Referring to FIGS. 1 and 2, the capsule 100 includes a housing having an elongated shape extending along an axis, which is also referred to in the following as “the longitudinal axis”. The elongated housing includes a cylindrical section and further include exterior rounded end portions, i.e. a proximal end portion and a distal end portion. In the shown embodiment an outlet 190 is arranged at a sidewall portion of the cylindrical section approximately midway between the proximal and distal end portions. The outlet thus points radially outwards from a surface arranged to be in close proximity with the tissue of the lumen wall. In the shown embodiment, the capsule is shaped in shape and size to roughly correspond to a 00 elongated capsule.

In the shown embodiment, the capsule 100 includes a drug outlet 190 that is positioned laterally to the longitudinal axis. The outlet 190 may be an aperture to permit jet injection to occur.

Existing jet injector systems for jet drug delivery are known in the art. A skilled person would understand how to select an appropriate jet injector that provides the correct jetting power to deliver the therapeutic substance into the lumen wall 24, for example from WO 2020/106,750 (PROGENITY INC).

In particular, the skilled person would understand that during drug delivery into a GI tract of a patient using jet injection, the jet stream created by the jet injector interfaces the lumen of the GI tract and the surface of the GI tract facing the lumen. Ultimately, the drug substance is deposited into the submucosal and/or the mucosal tissue by the substance impacting the mucosal layer of the GI tract (e.g. the epithelial layer and any mucus that may be present on the epithelial layer) as a stable jet stream of fluid with minimal breakup into a spray.

The volume of fluid of the drug substance experiences a peak fluid pressure that generates the jet stream that exits the jet injector with a peak jet velocity. The jet stream impacts the interface of the lumen of the GI tract and the surface of the GI tract facing the lumen with a peak jet power, peak jet pressure and peak jet force. The skilled person would recognise that these three parameters are interconnected.

The skilled person would understand how to assess and measure the various jet injector characteristics for suitability of use in the described type of jet injection. For example, one way to assess the jet power is to release the jets onto force sensors which measure the force the jet. Based on the force reading, and knowing the area of the nozzle and density of the jetted liquid, the jet velocity can be determined using equation 1. Based on the calculated velocity, the power (in Watts) can be calculated using equation 2. To evaluate the jet pressure (i.e. the pressure at which the jet stream is expelled), equation 3 can be used.

$$F = ρ {AV}^{2} \qquad ( equation 1 )$$

$$P = \frac{1}{2} ρ {AV}^{3} \qquad ( equation 2 )$$

$$V = \sqrt{\frac{2 * {P}_{bar} * 100000}{ρ * C}} \qquad ( equation 3 )$$

$$F = Force ( N )$$

$$ρ = Density ( kg / m 3 )$$

$$A = Area of nozzle ( m2 )$$

$$V = Velocity ( m / s )$$

$$P = power ( W )$$

$${P}_{bar} = Pressure ( bar )$$

$$C = Nozzle Loss Coefficient ( Typically 0.95 )$$

Referring to FIG. 1, the shown capsule 100 includes a main housing 110 defining a cylindrical sleeve member, and a generally cylindrical shaped core member 120 which is arranged within a cylindrical bore of main housing 110 and which extends axially along a major part of the main housing. In the shown embodiment, the core member 120 is fixedly mounted within the main housing 110. At the distal end of capsule 100, a cap 130 is attached which seals the main housing 110 at the distal end and which completely covers the core member 120. Within core member 120 a gas expansion unit is arranged, which in the shown embodiment comprises a pre-pressurized gas canister 150. The capsule further comprises a trigger arrangement which is configured to actuate the gas expansion unit, i.e., upon triggering by a predefined condition. When the capsule 100 is triggered, the gas expansion unit provides pressurized gas towards a liquid drug reservoir for expelling the liquid drug product contained within capsule 100 towards the outlet 190.

The cylindrical bore of main housing 110 is formed to be open at both ends. Hence, the main housing 110 at the proximal end provides an axial opening wherein the core member 120 is not covered by the main housing 110. However, the proximal end of capsule 100, and more specifically the proximal end of core member 120 is closed off by a semi-permeable membrane which serves as a fluid ingress port for gastrointestinal fluid and which forms part of a trigger arrangement for the capsule.

FIG. 2 shows a cross sectional view of the capsule 100 representing the assembled capsule in an initial state wherein the capsule is ready to be ingested by the patient. Inside capsule 100, at the proximal end thereof, core member 120 defines an elongated channel 121 which extends towards a larger diameter bore 122 arranged at the distal half of capsule 100. Inside the larger diameter bore 122 the pre-pressurized gas canister 150 is accommodated, the gas canister defining a gas pressurization chamber B. The axial length of larger diameter bore 122 is longer than the axial length of gas canister 150 allowing the gas canister to be moved axially from the initial proximal position (shown in FIG. 2) towards a second triggered position located more distally, i.e. at a distal end of capsule 100. The larger diameter bore 122 defines an actuation chamber A located distally from gas canister 150.

A drug reservoir is formed within capsule 100 as an elongated channel or duct with a particularly narrow cross-section compared to the length of the reservoir. In this disclosure the drug reservoir will be referred to as a capillary 125, or capillary duct, which is intended to accommodate a liquid drug product either during storage of the capsule 100, or becoming filled with the liquid drug product in the capillary immediately prior to the patient swallowing the capsule. In the embodiments shown herein, the capillary 125 forms a single capillary duct or channel between the gas expansion unit and the outlet.

For most embodiments, the capillary 125 forms an elongated channel having a total length which is larger than the axial length of the capsule 100, typically much larger than the axial length of capsule 100. In order to accomplish this the capillary 125 is arranged to extend along at least one non-rectilinear segment path, and typically through multiple non-rectilinear segments paths, from the capillary inlet section to the capillary outlet section, the latter being arranged adjacent the drug outlet 190 for fluid communication therewith. Hence, although the capillary 125 defines a narrow channel, the non-linear configuration of the capillary 125 serves to form a densely packed configuration providing a substantial volume for liquid drug to be accommodated in the capsule 100.

Referring to FIG. 2, in the shown embodiment, the capillary 125, i.e. the drug reservoir, is shaped to include a generally helically extending segment 125B, 125C leading from a distally arranged capillary inlet section 125A to a proximally arranged intermediary section 125D and further axially in a generally rectilinear shaped segment 125E, via radially outwards extending capillary outlet section 125F towards the drug outlet 190, arranged approximately midways axially between the capillary inlet section 125A and intermediary section 125D. In order for the rectilinear shaped segment 125E to axially cross the helically extending segment 125B, 125C, the rectilinear shaped segment 125E is arranged to extend radially inwards relative to the helically extending segment 125B, 125C in a radially overlapping manner.

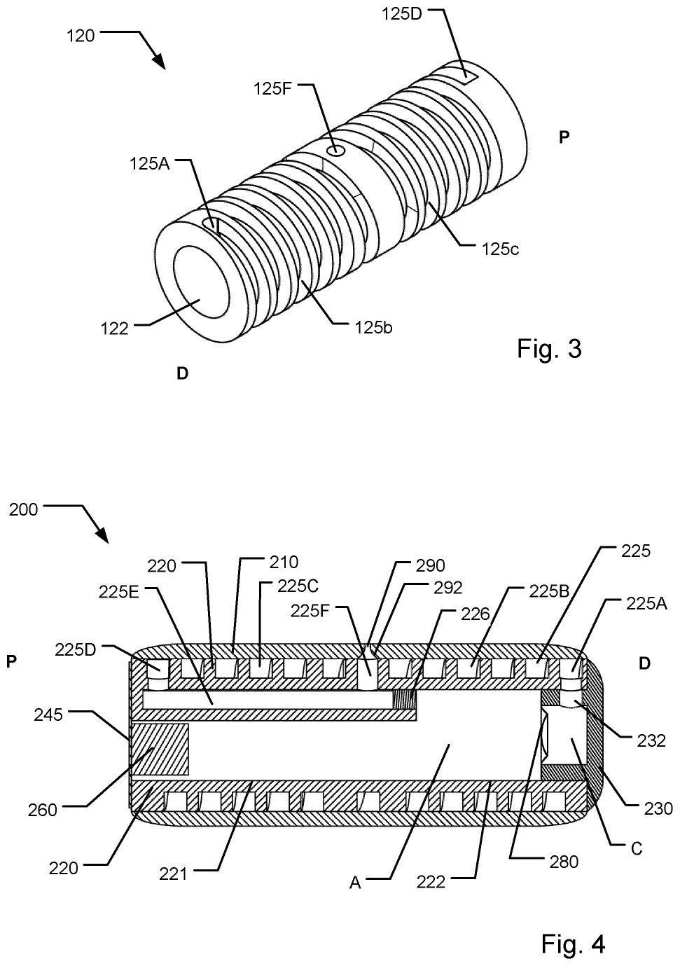

The core member 120, as separated from the remaining parts of capsule 100, is shown in a perspective exterior view in FIG. 3. In the shown embodiment, the core member 120 defines a generally cylindrical outer surface. The outer surface includes a generally helically extending recessed track 125b, 125c which extends from a distal end section of core member 120 to the proximal end section of core member. The capillary inlet section 125A located at the distal end of core member 120 extends as a radially inwards extending channel providing fluid communication between the larger diameter bore 122 and helical extending segment 125B, 125C. The intermediary segment 125D located at the proximal end of core member 120 extends as a radially inwards extending channel providing fluid communication between the helical extending segment 125B, 125C. The rectilinear shaped segment 125E extends axially distally from the intermediary segment 125D slightly axially past the drug outlet 190 where it is terminated by a plug 226. The radially outwards extending capillary outlet section 125F provides fluid communication between the rectilinear shaped segment 125E and a jet nozzle 192 formed in the cylindrical sleeve member of main housing 110, i.e., at drug outlet 190.

In order to accommodate the radially outwards extending capillary outlet section 125F, the helical extending segment is divided into two sub-segments 125B and 125C, respectively arranged proximally and distally relative to the capillary outlet section 125F. The pitch of the helical sub-segments 125B and 125C is selected so as to provide a densely packed capillary configuration. The channel interconnecting the two sub-segments 125B and 125C is formed with a pitch considerably larger in order to make room for the radially outwards extending capillary outlet section 125F.

In the shown embodiment, as shown in FIG. 2, the helically extending recessed track formed in the outer surface of core member 120 in combination with the circumferentially arranged outer sleeve of main housing 110 defines a capillary with a square cross-section defining corners having a relatively small radius of curvature. Such embodiment may provide a capsule optimized for lowering manufacturing costs. However, in other embodiments, the capillary may be formed with differently shaped cross-section, such as square cross-sections having corners with a larger radius of curvature. Also, in particular embodiments, the capillary may be formed to provide a cross-sectional shape defining a circle. It is to be noted though, that the capillary does not need to have the same cross-sectional shape or dimension throughout the extension of the capillary 125, i.e., throughout segments/sections 125A, 125B, 125C, 125D, 125E and 125F, but may be formed with differing shape and/or varying area.

In accordance with an aspect of the present invention, expulsion of drug from the drug reservoir, i.e., the capillary 125, is performed without the use of a separating member, such as a piston or sealing plunger, arranged between an expanding gas and the liquid drug accommodated in the reservoir. Instead, the capillary 125 is designed, in combination with the properties of the liquid and the gas expansion unit, so that the liquid drug accommodated in the capillary exhibits a well-defined liquid interface relative to an expanding gas that acts on the liquid towards emptying the liquid drug through the outlet 190. A well-defined liquid interface means that, although the gas acts directly on the liquid interface, no mix-up, or only a non-substantial mix-up, of gas from the gas expansion unit and liquid drug occurs during storage and/or during emptying of liquid drug from the capillary 125. In this way no droplets of liquid, or only an insignificant number of droplets, will remain in the capillary at locations where the pressurized gas has evacuated the liquid. Likewise, no or only an insignificant amount of gas bubbles will enter into the liquid column in the capillary 125. In the course of drug expelling, the liquid interface, i.e., the trailing end of the liquid column, travels through the capillary 125 towards the drug outlet and will eventually reach the jet nozzle 192.

The outlet 190 arranged at the end of capillary 125 defines a fluid outlet passage from the reservoir to the exterior of the capsule 100. In the shown embodiment, the outlet 190 includes a jet nozzle 192 dimensioned and shaped to create a liquid jet stream of drug when the drug is forced through the outlet. The reservoir may be sealed at the outlet with a seal designed to break at high pressure of the liquid drug.

When the capsule assumes an initial state, i.e., prior to administration, a liquid drug substance is accommodated in the reservoir, i.e., within capillary 125. In the embodiment shown, the liquid drug is filled so that the liquid completely fills the capillary 125 all the way from capillary inlet section 125A to capillary outlet section 125F, and possibly even filling the interior space defined by jet nozzle 192, so that the liquid contained in capsule 100 forms a contiguous volume of liquid with no bubbles of gas, such as air. In the initial state, the liquid interface may be located at channel 132.

Referring again to FIG. 2, the capillary 125 is arranged in fluid communication with the actuation chamber A via a channel 132 formed in cap 130. In the shown embodiment, the cap 130 further includes a spike 170 formed unitarily with cap 130.

As mentioned above, in the shown embodiment, the gas expansion unit includes a pre-pressurized gas cannister 150. Gas cannister 150 forms an enclosure having a cylindrical space B which accommodates a gas stored at high pressure. The cylindrical space is closed by a rupturable seal 151 which in this embodiment is provided as a membrane made from a thin foil material, e.g., aluminium foil, the rupturable seal 151 facing towards the distal end of capsule 100. In this first embodiment the gas cannister 150 is arranged axially slideable within the larger diameter bore 122.

The spike 170 is fixedly disposed onto the cap 130 at a central location thereof, i.e., arranged coaxially with the longitudinal axis. The spike has a pointed end pointing in the proximal direction and hence towards rupturable seal 151 of gas cannister 150. The spike 170 is configured to rupture seal 151 upon gas canister 150 being moved distally relative to spike 170 to allow pressurized gas to escape cylindrical space B and flow into the actuation chamber A.

As noted above the proximal end of core member 120 is closed off by a semi-permeable membrane 145 which serves as a fluid ingress port and which forms part of the trigger arrangement for the capsule. Semi-permeable membrane 145 is fixedly arranged onto a proximally facing end wall of core member 120 and so that semi-permeable membrane covers the proximal end of the elongated channel 121 formed within core member 120. Hence, gastrointestinal fluid that enters into elongated channel 121 of the capsule 100 needs to pass through the semipermeable membrane 145. The proximally facing end wall of core member 120 provides sufficient structural strength and area to serve as a mounting surface for semi-permeable membrane 145.

For the shown embodiment capsule 100 example materials for the semi-permeable membrane 145 may be made from Standard Grade Regenerated Cellulose (RC). The material for the semi-permeable membrane 145 may be selected so that it is biodegradable when subjected to biological fluid.

A piece of a sponge material 140 is arranged in proximity of the semi-permeable membrane 145, such as in abutting relationship. Sponge material 140 may be formed by absorbent material made from a fibrous, porous or microporous, open-celled material chosen to exhibit a marked ability to rapidly swell when being subjected to contact with a liquid. In the shown embodiment the sponge portion 140 is a dry cellulose sponge provided in compressed form, wherein the cellulose is provided as a biodegradable sponge.

The sponge portion 140 is arranged in the elongated channel 121 disposed axially between the semi-permeable membrane 145 and the gas cannister 150. To enable the semi-permeable membrane to quickly soak in gastric fluid through openings 115, i.e. to serve in combination with the semi-permeable membrane as an osmotic drive, a salt 142 or similar material is positioned in contact with both the semi-permeable membrane 145 and the sponge portion 140. In the shown embodiment the semi-permeable membrane 145, the sponge portion 140 and the gas canister 150 may be adhered to each other with the salt 142 arranged in a cavity formed in the sponge portion 140. For some embodiments, the sponge portion 140 may be constrained around its circumference so that the sponge primarily or exclusively expands in the axial dimension as gastrointestinal fluid makes the sponge expand.

In the shown embodiment, the semi-permeable membrane 145, the salt 142, the sponge portion 140 and the spike 170 in combination forms a trigger assembly. Also, in the shown embodiment, although not visible in FIGS. 1 and 2, the semi-permeable membrane 145 is initially covered by a layer of a pH-sensitive enteric coating which initially blocks fluid ingress through the semi-permeable membrane 145. As known in the art, the enteric coating may be configured to utilize the marked shift in pH-level that the capsule 100 experiences when travelling from the stomach to the small intestine. After the capsule has entered the small intestine, after a predefined time, the enteric coating will become sufficiently degraded so that gastrointestinal fluid may enter through the semi-permeable membrane 145.

Next the operation of capsule 100 will be described. Subsequent to the patient swallows capsule 100, upon entering the small intestine, the enteric coating of the capsule 100 will begin dissolving and gastric fluid will soon after be available for the osmotic drive to provide fluid transport across the semi-permeable membrane 145.

As gastrointestinal fluid gets into contact with sponge portion 140 the sponge rapidly starts to expand. In the shown embodiment, the sponge portion 140 may be constrained around its circumference so that the sponge primarily or exclusively expands in the axial dimension as fluid makes the sponge swell. Axial swelling of the sponge portion 140 causes the gas canister 150 to displace distally in the course of fluid ingress through the semi-permeable membrane 145.

As the gas canister 150 moves distally, the spike 170 will start contacting the rupturable seal 151. Upon further distal movement of gas canister 150, the spike 170 will at some point penetrate the rupturable seal 151, whereafter the pressurized gas within gas canister will escape to the actuation chamber A and will rapidly increase the gas pressure so as to act directly on the liquid drug interface. As noted above, depending on the filling level of the capsule from the outset, the liquid interface may initially either be provided inside capillary 125, such as within capillary inlet section 125A, or within channel 132.

The rapid increase in gas pressure in actuation chamber A exerts load, i.e. elevated gas pressure, directly on the liquid drug interface acting to push the entire liquid column present in the capillary 125 towards the outlet and a stream of liquid jet starts to form from jet nozzle 192. The power of the jet stream is configured to penetrate the mucosal tissue so as to form a drug depot within the tissue of the lumen wall of the small intestine.

Eventually, all the liquid drug present in capillary 125 will be emptied from capillary 125 and the jet stream of drug through the jet nozzle 192 will end. After delivery of the liquid drug, the capsule 100 is allowed to pass the alimentary canal and be subsequently excreted.

Referring now to FIG. 4, a second embodiment of a capsule 200 will now be described. The capsule 200 corresponds in many aspects to the capsule 100 but the triggerable expelling system, i.e. the gas expansion unit and the trigger arrangement, is different.

Main housing 210 and core member 220 of the second embodiment are formed similarly to main housing 110 and core member 120 of the first embodiment. Hence capillary duct 225, i.e. the drug reservoir, and the drug outlet 290 corresponds in structure and function to that of the capsule 100.

A distally arranged cap 230 again seals off distal end of capsule 200. Cap 230 also provides a channel 232 so that the capillary 225 is arranged in fluid communication with an intermediate chamber C via a channel 232 formed in cap 230.

For the second embodiment capsule 200, an expelling system is arranged configured for generating a pressurized gas upon triggering, i.e. upon triggering by a predefined condition. The drive system comprises a gas generator capable of producing a gas for exerting load on the liquid column in capillary 225 but only subject to elevated gas pressure from the gas generator exceeding a predefined threshold. In the shown embodiment, the gas generator is arranged inside a hollow space of core member 220 defining an actuation chamber A, i.e. elongated channel 221 and larger diameter bore 222.

Gas may be generated by chemical reaction so that once the gas generator is actuated gas is produced to form pressurized gas in the actuation chamber A of capsule 200. Different principles may be used for providing gas generation inside the actuation chamber A, for example by using a gas producing cell, such as a hydrogen cell, an airbag inflator, a gas generator utilizing phase change, or a generator which incorporates mixing of reactants to chemically react to form a gas, such as by mixing sodium bicarbonate and acid. For gas generation using mixing of reactants, either all reactants may be stored on board the capsule prior to actuation, or at least one reactant may be introduced into the capsule for mixing with a reactant stored on board the capsule.

The following are examples of chemical reactions which produce carbon dioxide CO₂ and which may be used as the components for generating pressurized gas in the actuation chamber A:
Example 1 (calcium carbonate with hydrochloric acid):CaCo3+2HCl→CaCl2)+H2O+CO2
Example 2 (citric acid with sodium bicarbonate):C6H8O7+3NaHCO3→3H2O+CO2+Na3C6H5O7
Example 3 (tartaric acid with sodium bicarbonate):H2C4H4O6+2NaHCO3→Na2C4H4O6+2H2O+2CO2

Examples of acids for effervescent reaction:
Citric acid
Acetic acid
Hydrochloric acid
Tartaric acid
Malic acid
Adipic acid
Ascorbic acid
Fumaric acid

Examples of carbonate salts for effervescent reaction:
Sodium bicarbonate
Sodium carbonate
Calcium carbonate
Potassium bicarbonate

In other embodiments, the effervescent reaction may occur by one or more solid state components being wetted (e.g. exposed to intestinal fluid or other fluid stored in capsule 200) which causes the effervescent reaction.

In capsule 200 shown embodiment in FIG. 4, gas is generated in the actuation chamber A by means of an internally arranged effervescent material 260 arranged in the actuation chamber, and by means of a semi-permeable membrane 245 which serves to introduce gastrointestinal fluid into the actuation chamber A to react with the effervescent material portion 260.

Effervescent material portion 260 may be formed from powder components that are subsequently compressed into block-shape. The block-shaped effervescent material portion 260 includes in this embodiment an effervescent couple comprised of at least one acidic material and one basic material, such as sodium bicarbonate and citric acid. The block of effervescent material 260 is adhered to semi-permeable membrane 245 to ensure close proximity with the membrane while leaving a volume of actuation chamber A available for gas generation.

As noted above in connection with the first embodiment capsule 100, the proximal end of core member 220 of the second embodiment capsule 200 is closed off by a semi-permeable membrane 245 which serves as a fluid ingress port and which forms part of the trigger arrangement for the capsule. Semi-permeable membrane 245 is fixedly arranged onto a proximally facing end wall of core member 220 so that semi-permeable membrane covers the proximal end of the elongated channel 221 formed within core member 220. Hence, gastrointestinal fluid that enters elongated channel 221 of the capsule 200 needs to pass through the semi-permeable membrane 245. The proximally facing end wall of core member 220 provides sufficient structural strength and area to serve as a mounting surface for semi-permeable membrane 245. In other embodiments, the membrane may be mounted relative to the core member by means of a clamping structure.

For the shown embodiment capsule 200 example materials for the semi-permeable membrane 245 may be made from Standard Grade Regenerated Cellulose (RC). The material for the semi-permeable membrane 245 may be selected so that it is biodegradable when subjected to biological fluid.

A burst member serving as a burst gate is arranged axially between the actuation chamber A and intermediate chamber C. The burst member functions as a gate to release load provided by the pressurized gas onto liquid drug column in capillary 225 but only upon increase in gas pressure in the actuation chamber A above a predefined threshold pressure level. For gas pressures below the predefined threshold pressure level, the burst member forms a substantially gas tight seal preventing liquid drug accommodated in capillary 225 to be moved towards the outlet 290.

In the shown embodiment capsule 200 includes a burst gate in the form of a rupturable membrane 280 which is mounted axially fixed at an axial location adjacent to distal end cap 230. Different attachment methods may be used for mounting the rupturable membrane 230 in capsule 200, such as by being adhered relative to a housing portion, or by clamping of the burst membrane between rigid structures mounted fixedly relative to one or more housing portions.

In the FIG. 4 embodiment, the rupturable membrane 280 is formed as a thin planar disc. Example materials for the rupturable membrane may be selected from a metallic material, such as aluminium, a polymer material or other suitable material that will exhibit a well-defined ability to burst at the predetermined threshold pressure level. Instead of forming the rupturable membrane as a planar disc, the burst gate may include forms of thin-layered material which in the initial state may exhibit or comprise one or more convex and/or concave portions.

In the example capsule 200 shown in FIG. 4, the jet delivery may be dimensioned to operate at a maximum fluid pressure in the order of 12 bar in the actuation chamber B. In the example shown the semi-permeable membrane 245 will be able to withstand a maximum gas pressure slightly above 12 bar before leaking. In accordance herewith, the burst disc may be designed to provide a release of gas towards interface of the liquid drug substance when the gas pressure level exceeds 12 bar.

The rupturable membrane 280 may in different embodiments include scoring lines or other weakened portions which define the location or locations wherein the rupturable membrane will initiate breaking when gas pressure exceeds the predetermined threshold pressure level.

In the second embodiment capsule 200, although not visible in FIG. 4, the semi-permeable membrane 245 is initially covered by a layer of a pH-sensitive enteric coating which initially blocks fluid ingress through the semi-permeable membrane 245. As known in the art, the enteric coating may be configured to utilize the marked shift in pH-level that the capsule 200 experiences when travelling from the stomach to the small intestine. After the capsule has entered the small intestine, after lapse of a predefined time, the enteric coating will become sufficiently degraded so that gastrointestinal fluid may enter through the semi-permeable membrane 245 and start migration of fluid through the membrane towards the effervescent material portion 260. For the shown embodiment in FIG. 4, the enteric coating forms part of a trigger arrangement for actuating the gas generator formed by the semi-permeable membrane 245 and the effervescent material portion 260.

Next the operation of capsule 200 will be described. Subsequent to a patient swallows capsule 200, upon entering the small intestine, the enteric coating of the capsule 200 will begin dissolving and gastrointestinal fluid will soon after be available enabling fluid transport across the semi-permeable membrane 245.

As fluid gets into contact with the effervescent material portion 260 pressurized gas will start to form in the actuation chamber A whereby gas pressure will gradually increase and provide an increasing load on the rupturable membrane 280. Subsequent to lapse of a predefined time period the gas pressure level in actuation chamber A exceeds the predetermined threshold pressure level which will cause the rupturable membrane 280 to burst.

Hereafter the pressurized gas within actuation chamber A will escape through the ruptured membrane 280 and gas pressure inside intermediate chamber C will rapidly increase. The rapid increase in gas pressure in intermediate chamber C exerts load directly on the liquid drug interface acting to push the entire liquid column present in the capillary 225 towards the outlet and a stream of liquid jet starts to form from jet nozzle 292. The power of the jet stream is configured to penetrate the mucosal tissue so as to form a drug depot within the tissue of the lumen wall of the small intestine.

Eventually, all the liquid drug present in capillary 225 will be emptied from capillary 225 and the jet stream of drug through the jet nozzle 292 will end. After delivery of the liquid drug, the capsule 200 is allowed to pass the alimentary canal and be subsequently excreted.

As described in the above embodiments, subsequent to swallowing, the capsule device first moves through the stomach and subsequently enters the small intestine. Due to the enteric coating becomes dissolved when entering the small intestine, the fluid ingress into capsules 100 and 200 will only be initiated upon the enteric coating becoming sufficiently dissolved for fluid ingress through the fluid inlet/semi-permeable membrane is enabled.

An enteric coating may be any suitable coating that allows the coated object to be activated for release in the intestine. In some cases, an enteric coating may dissolve preferentially in the small intestine as compared to the stomach. In other embodiments, the enteric coating may hydrolyse preferentially in the small intestine as compared to the stomach. Non-limiting examples of materials used as enteric coatings include methyl acrylate-methacrylic acid copolymers, cellulose acetate succinate, hydroxy propyl methyl cellulose phthalate, hydroxy propyl methyl cellulose acetate succinate (i.e., hypromellose acetate succinate), polyvinyl acetate phthalate (PVAP), methyl methacrylate-methacrylic acid copolymers, and sodium alginate, and stearic acid. Additional examples are disclosed in e.g., US 2018/0193621 hereby incorporated by reference. A given object (here: capsule), or a fluid inlet only, may be coated with an enteric coating. The enteric coating may be composed to be soluble at a given pH or within a given pH range, e.g. at a pH greater than 5.5, at a pH greater than 6.5, within a range of about 5.6 to 6 or within a range of about 5.6 to 6.5 or 7. The dissolution time at an intestinal pH may be controlled or adjusted by the composition of the enteric coating. For example, the dissolution time at an intestinal pH may be controlled or adjusted by the thickness of the enteric coating.

In other embodiments, the condition for controlling when triggering is to occur may be provided by means of other principles. For example, a dissolvable layer may be disposed initially blocking the fluid inlet of the capsule, with dissolution of the dissolvable layer being initiated at first exposure to gastric fluid, with the timing of the dissolvable layer being decisive for the location at which the capsule deploys. Also, such as for a stomach deployable capsule, no coating may be present, so that the triggering of the gas expansion unit occurs as soon as sufficient liquid has been transferred through the semi-permeable membrane. Still other triggering principles may rely on temperature change induced passage of gastric fluid though the fluid inlet and into the capsule gas expansion unit.

Although the above description of exemplary embodiments mainly concerns ingestible capsules for delivery in the small intestine, the present invention generally finds utility in capsule devices for lumen insertion in general, wherein a capsule device is positioned into a body lumen for delivery of a drug product. Non-limiting examples of capsule devices include capsule devices for delivery in the stomach or delivery into the tissue of the stomach wall. For example, various self-righting or self-orienting structures and/or methods described in WO 2018/213600 can be employed by the capsule device in accordance with the present disclosure. WO 2018/213600 is incorporated herein by reference in its entirety.

In various embodiments of capsules utilizing the specific drug reservoir and expelling arrangement described herein, drug delivery may be performed using a delivery member, such as a needle, via a jet stream of liquid to provide needle-free liquid jet penetration into the mucosal lining or via spraying inside the lumen.

As disclosed herein, the capillary ducts 125 and 225 may be formed by making suitable recessed portions in first and second parts which in combination, when assembled, forms the desired capillary duct. Although in the first and second embodiments the recessed tracks are formed in a core member 120/220, the recessed tracks may instead be formed in the main housing 110/210. In still other embodiments, both parts may include recessed areas which combines when assembled to form a capillary duct having the desired cross section. For example, each of a first and a second member may include a recessed track which may be formed with a half-circular recess into a surface. When the first and second parts are assembled the half-circular recessed track of the first part and the half-circular recessed track of the second part will in combination form a capillary duct having a circular cross-section.

Also, in other embodiments, the capillary may be formed with other cross-sectional shapes such as oval or polygonal. Embodiments may be provided wherein the capillary duct has a rectangular cross section wherein the cross section of the capillary may be formed as a thin slot which is relatively wide in directions laterally to the thickness dimension. Still other embodiments may include a first and second coaxially arranged cylinders, e.g. a second cylinder arranged to circumscribe the first cylinder, wherein a thin cylindrical gap is formed between the first and second cylinder, i.e. so that the capillary defines a ring-shaped circular cross section, the cylindrical cross section circumscribing the first cylinder.

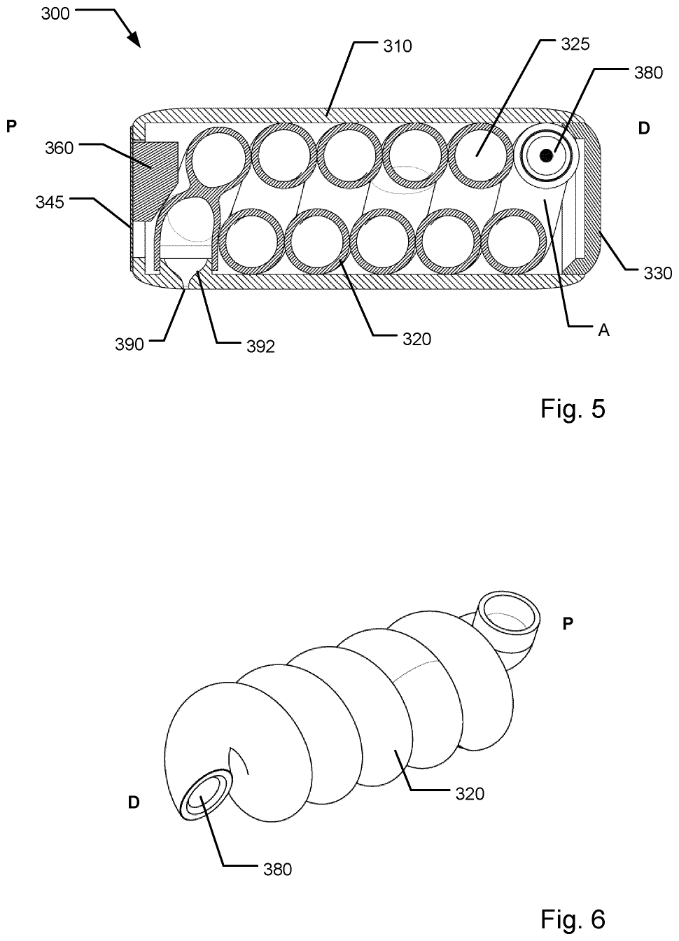

Referring to FIGS. 5 and 6 a further third embodiment capsule 300 will next be described. The capsule 300 is designed to work similarly to capsule 200 described above, but the capillary duct has been designed differently. Whereas the capillary 225 is made from first and second parts that in combination forms the capillary 225, the capsule 300 includes a single entity capillary 325 made from moulded portions that are joined to a form a single member tubing 320. The single member tubing 320 is subsequently inserted into the capsule housing 310. The tubing 320 may either be formed from a rigid material or, alternatively from a flexible material, and the tubing is arranged to extend along a helical path, in the shown embodiment with approximately 5.5 windings from a first inlet end to a second outlet end.

A burst gate is provided at the first inlet end of tubing 320 in a manner fixedly attached to the tubing 320. The burst gate is again provided in the form of a rupturable membrane 380 which prior to triggering of capsule 300 forms a liquid tight seal at the inlet end of tubing 320. The rupturable membrane 380 is configured to release load, i.e., gas pressure, onto liquid drug substance in capillary 325 upon increase in gas pressure in the actuation chamber (A) above a threshold pressure level to thereby initiate expelling of the drug substance.

As shown in FIG. 5, with the tubing 320 arranged in the capsule housing 310, the second outlet end of the tubing 320 is mated in a sealing manner relative to drug outlet 390, however with the drug outlet being located at the proximal end of the capsule housing 310, but again pointing radially outwards relative to the lateral surface of the elongated capsule. Similar to the second embodiment, although not shown in the drawing, a removable seal may be arranged to seal of the drug outlet 390, either at the output side of jet nozzle 392 or at an internal position upstream from jet nozzle 392. Inside tubing 320, between the first inlet end and the second outlet end, a liquid drug substance is stored.

Also for the third embodiment capsule 300 an expelling system is arranged configured for generating a pressurized gas upon triggering, i.e., upon triggering by a predefined condition. The drive system comprises a gas generator capable of producing a gas for exerting load on the liquid column in capillary 325 but only subject to elevated gas pressure from the gas generator exceeding a predefined pressure threshold. Again, the expelling system incorporates a semipermeable membrane 345 similar to membrane 245 and effervescent material portion 360 similar to effervescent material portion 260 of the second embodiment. The effervescent material portion is arranged at the proximal end of capsule housing 310 and the tubing 320 and capsule housing 310 are designed so that gas generated at the proximal end of capsule housing will flow unhindered towards the rupturable membrane 380. Again, as for the second capsule 200, a distal end cap 330 is mounted in a sealing manner to the distal end of the capsule housing 31. A suitable triggering arrangement is included but not shown.

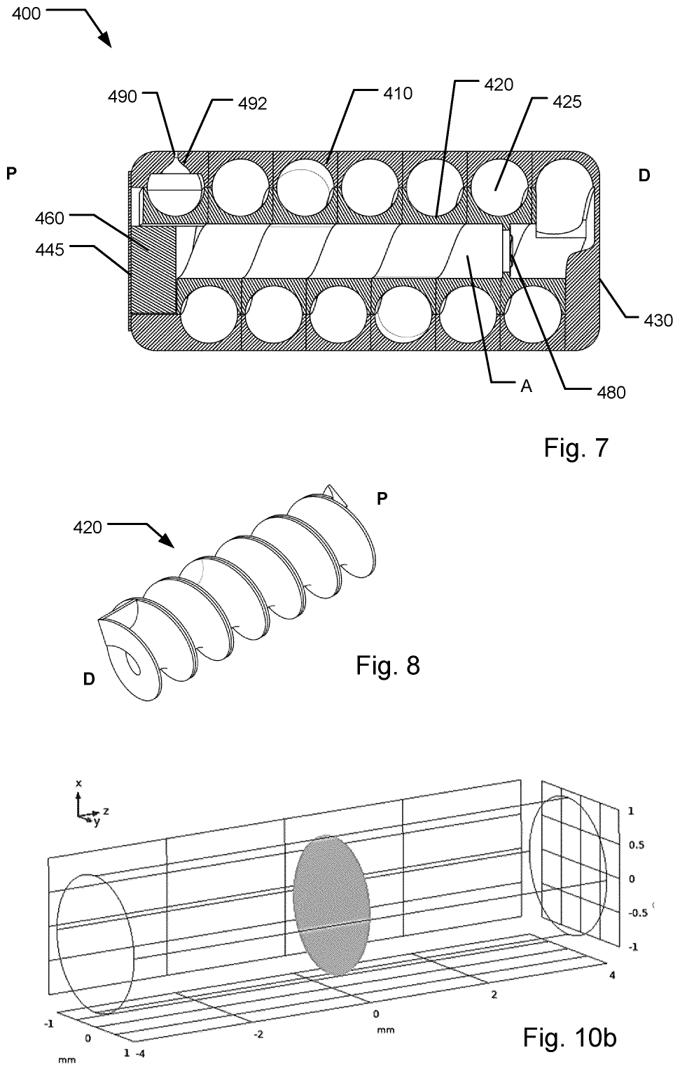

A fourth embodiment capsule 400 is shown in FIGS. 7 and 8. The capsule 400 is designed to work similarly to capsule 300 described above, but the capillary duct has again been designed differently. Capsule 400 is provided with a capsule housing 410 having an elongated, general cylindrical shape, with a smooth outer surface, but with a radially inwards facing surface which is shaped to form part of a helical extending capillary duct 425. The radially inwards facing surface includes a recessed track which is formed with a half-circular recess. A core member 420 includes a recessed track which is formed as a half-circular recess into a radially outwards facing surface. When the core member 420 is inserted into the capsule housing 410, the half-circular recessed track of the core member and the half-circular recessed track of the capsule housing in combination provide a capillary duct having a circular cross-section arranged to extend along a helical path from a first inlet end to a second outlet end. In the shown fourth embodiment the helical capillary duct 425 is formed with approximately 6 windings from the first inlet end to the second outlet end.

A burst gate provided as a rupturable membrane 480 is provided “upstream” from the first inlet end of capillary 425. In the shown embodiment, the rupturable membrane 4808 is fixedly mounted within a longitudinally extending through-going bore of the core member 420, at a distal end thereof.

As shown in FIG. 7, a drug outlet 490, shaped integrally within capsule housing 410, is formed with a jet nozzle 492. As the capsule housing 410 partially constitutes the capillary duct 425 no joining between the capillary duct 425 and drug outlet is needed. The drug outlet is located at the proximal end of the capsule housing 410 in a manner pointing radially outwards relative to the lateral surface of the elongated capsule to enable liquid jet penetration of tissue encircling the capsule 400. Similar to the third embodiment, although not shown in the drawing, a removable seal may be arranged to seal of the drug outlet 490, either at the output side of jet nozzle 492 or at an internal position upstream from jet nozzle 492. Inside capillary duct 425, between the first inlet end and the second outlet end, a liquid drug substance is stored.

Similar to the third embodiment, the fourth embodiment capsule 400 comprises an expelling system configured for generating a pressurized gas upon triggering, i.e., upon triggering by a predefined condition. The drive system comprises a gas generator capable of producing a gas for exerting load on the liquid column in capillary 425 but only subject to elevated gas pressure from the gas generator exceeding a predefined pressure threshold. Again, the expelling system incorporates a semipermeable membrane 445 similar to membrane 345 and effervescent material portion 460 similar to effervescent material portion 360 of the third embodiment. The effervescent material portion is arranged at the proximal end of capsule housing 410/core member 420 so that gas generated at the proximal end within the longitudinally extending bore of core member 420 can flow unhindered towards the rupturable membrane 480.

In the fourth embodiment, a distal end cap 430 is formed integrally with the capsule housing 410. A suitable triggering arrangement is included but not shown.

In the following different parameters decisive for the capsule device operation and the capillary expelling function will be discussed.

### Example 1

For an example capsule device according to the invention, a suitable jet nozzle size Do may be selected around 0.25 mm, and applied pressure p in the ampoule may be chosen around 10 bar, this being determined by the jet injection process.

Target for the nozzle design is to create a jet that delivers power P=p Q of around 2 W, i.e., flow rate Q=P/p is around 2000 mm³/s.

If the diameter D₁ of the reservoir or “capillary” is small then it also needs to be long to accommodate a given drug volume, which can make it difficult to fit inside a device to be swallowed.

Other constraints or design considerations may include the flow resistance in the reservoir section so as not to cause significant pressure loss.

Additionally, the system needs to be designed such that surface tension forces can maintain the liquid/gas interface well-defined and ensure that all the drug is expelled from the capillary.

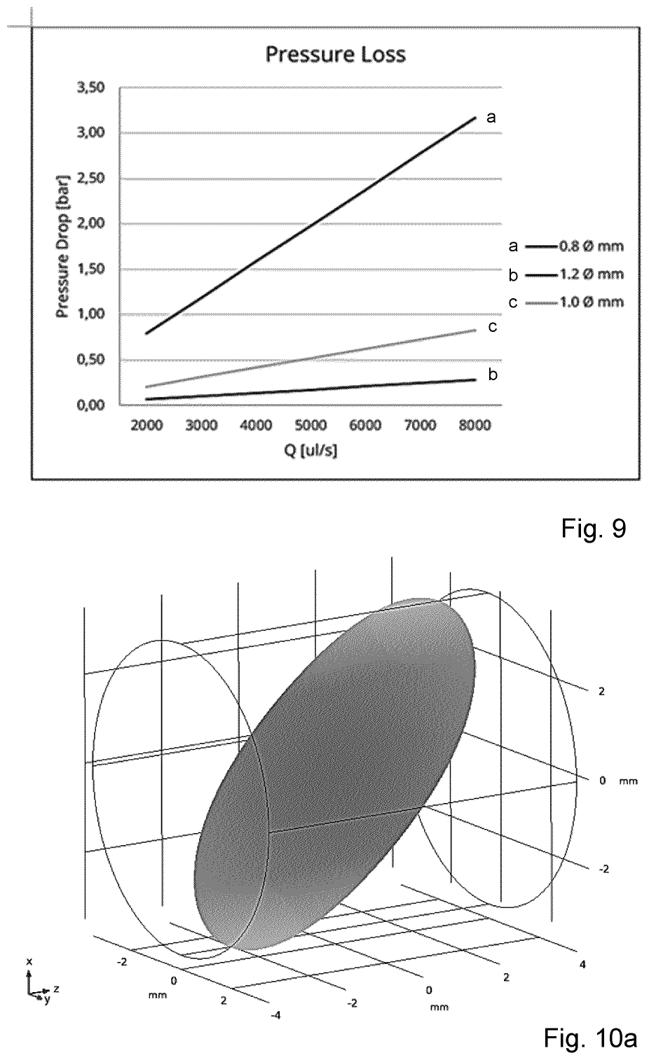

The flow resistance in the reservoir is

$${R}_{hyd} = \frac{128 η {L}_{1}}{π {D}_{1}^{4}} \qquad ( equation 4 )$$

where the L₁ is the length of the liquid column in the reservoir. To accommodate a drug volume V we need L₁=V/A₁ where A₁=πD₁²/4 is the cross-sectional area. The pressure loss is Δp=R_{hyd}·Q and for a water-like drug with viscosity η=0.001 Pa s and volume V=100 mm³, expelled at Q≈2000 mm³/s, the pressure loss is <0.4 bar when the reservoir diameter is D₁>0.8 mm, cf. FIG. 9.

In order for the surface tension to overcome gravity and maintain a well-defined interface in the reservoir, the reservoir diameter needs to be small or not-so-large compared with the socalled “capillary length” l_{c}=\sqrt{γ/μg}. For at drug with surface tension γ=0.050 N/m, density ρ=1000 kg/m³, and gravity g=9.8 m/s² we have l_{c}=2.3 mm. Hence, the reservoir for such system cannot be much larger than 2.3 mm.

Simulation indicates the interface becomes unstable when the ratio D₁/l_{c}>1.55, i.e., with l_{c}=2.3 mm that corresponds to D₁=3.57 mm. FIG. 10a shows liquid/gas interface at D₁=3.5 mm whereas FIG. 10b shows liquid/gas interface at D₁=1.0 mm both with contact angle θ=90° and gravity pointing “down”. For D₁>3.57 mm the liquid may form a puddle at one side of the reservoir.

If the device is shaken or dropped then the g-force is >9.8 m/s² and the interface may be disturbed in any case. This is unlikely to occur during the jet injection event, but during storage either the reservoir should be free of air or we need to consider how the functionality is affected if the air forms bubbles in the liquid column or even in the nozzle region.

In order not to leave droplets of drug on the reservoir surface it is necessary to choose a material that is moderately hydrophobic with contact angle θ towards drug of >>0°. Ideally around θ≈90° will be obtainable for a polymer reservoir.

The characteristic velocity for balance between viscous forces and surface tension at the moving interface is v*=γ/η. For a drug with γ=0.050 N/m and η=0.001 Pa s we get v*=50 m/s. The ratio of the interface speed v₁=Q/A₁ to the characteristic velocity is the socalled “capillary number”

$$Ca = \frac{{v}_{1} η}{γ} = \frac{{v}_{1}}{{v}^{*}} \qquad ( equation 5 )$$

When Ca number is small, the surface tension is able to maintain the interface intact and empty the reservoir completely. However, at larger Ca the hydrodynamics at the moving interface leaves behind a liquid film that will subsequently form droplets on the surface.

The relation between film thickness and Ca has been investigated by Bretherton (1961) and more recently Giavedoni (1997).

The transition between full recovery of the drug and droplet formation occurs at a critical velocity of

$${v}_{max} \approx {v}^{*} \cdot \frac{{θ}^{3}}{312} \qquad ( equation 6 )$$

$$\frac{{v}_{1}}{{v}_{max}} = \frac{{v}_{1}}{{v}^{*}} \cdot \frac{312}{{θ}^{3}} = Ca \cdot \frac{312}{{θ}^{3}} \qquad ( equation 7 )$$

where θ is the contact angle in radians, (cf. p. 143 in “P.-G. de Gennes; F. Brochard-Wyart; D. Quéré in Capillarity and wetting phenomena; Springer: New York; 2004”). With θ=90°=1.57 rad and v*=50 m/s we get vₘₐₓ≈0.6 m/s.

In order to maintain a flow rate of Q=2000 mm³/s we therefore need the reservoir area to exceed A₁>Q/vₘₐₓ≈3.3 mm² or D₁>2.1 mm.

It appears there is a window with D₁>2.1 mm but not greatly exceeding l_{c}=2.3 mm.

If the drug is (significantly) more viscous then v* and vₘₐₓ is lowered and the window is more narrow, likewise if the reservoir surface is more hydrophilic and θ is small then vₘₐₓ is lowered and we cannot accommodate as high a flow rate with the same reservoir diameter.

If we allow the nozzle design to be a factor, we could reach the same power P=p·Q=½ρv₀². Q=½ρv₀³A₀ by increasing p and decreasing the nozzle area and thereby Q.

However, that is not so attractive because it implies higher energy loss: E=P·Δt=p·V. For a given drug volume we want to run the jet injection at the lowest possible pressure to minimize the size of the energy source driving the flow.

### End-of-Example 1

Identifying Experimental Parameters

To best identify experimental parameters using the equations above, a step-by-step demonstration below using graphs will provide guidance.

Which drug and capillary material to be used? Knowing the specific drug, one can know the surface tension of the drug, and knowing the capillary material, one can identify the contact angle. Once these are identified in addition to viscosity of drug, the following equation can be used to calculate the max velocity the drug can be driven/jetted without disrupting the interface (meaning, without gas flowing throw the drug resulting in spray)

$${v}_{max} \approx \frac{γ}{η} \cdot \frac{{θ}^{3}}{312} \qquad ( equation 8 )$$

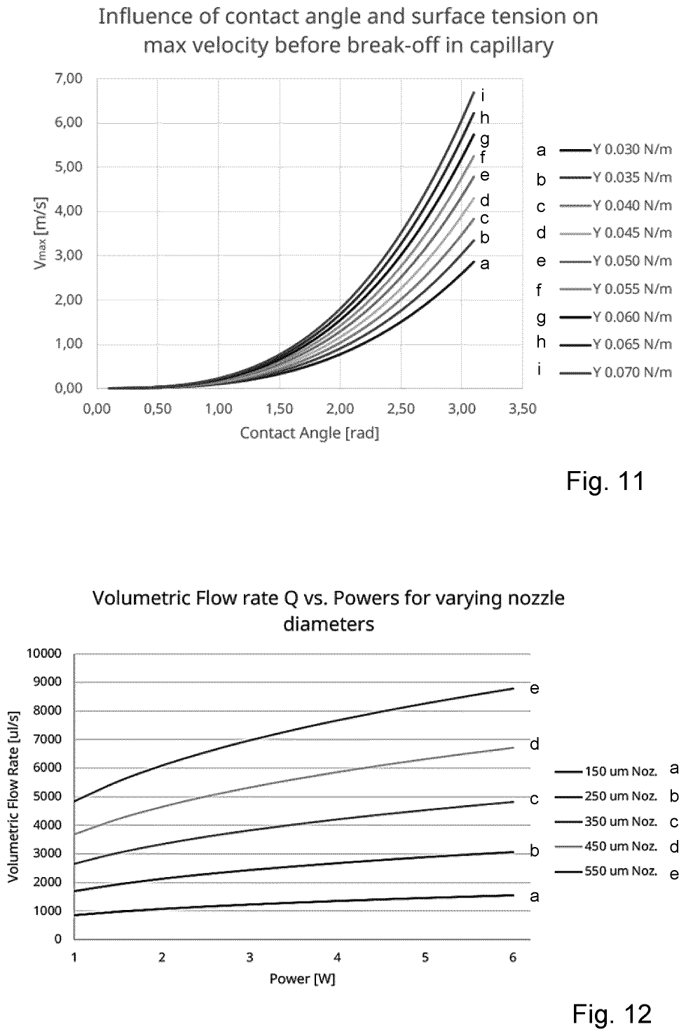

FIG. 11 is a graph showing Influence of contact angle and surface tension on max velocity before break-off in capillary.

Once Vₘₐₓ in capillary is calculated, one must identify which nozzle diameter to use, which in turn affects the area of the nozzle, which in turn affects the volumetric flow rate (Q) of ejected jet since Q=vₘₐₓ*A where A is area of nozzle which leads to identifying the diameter of the capillary. The Diameter of capillary varies depending on the desired jet Power shown in the equation below:

$$P = \frac{1}{2} ρ {v}^{3} A \qquad ( equation 9 )$$

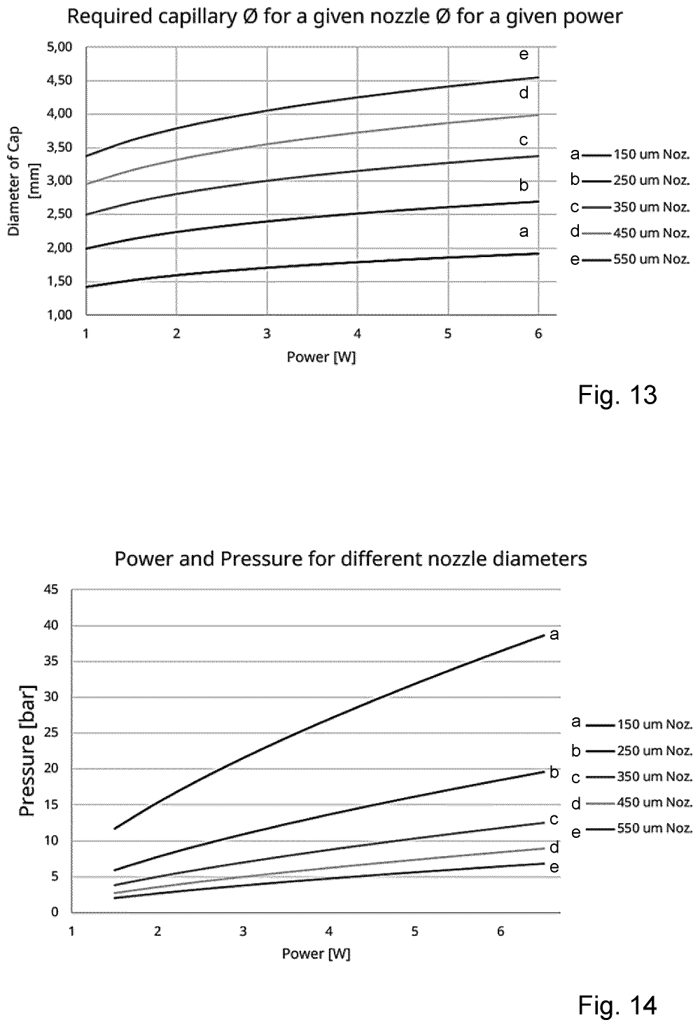

These correlations can be seen in FIGS. 12 and 13, wherein given a desired nozzle diameter and jet power, the diameter of a capillary can be identified, all the while ensuring a stable drug interface in the capillary.

Volumetric flow rates can also be expressed in terms of gas pressure for a given desired Power since P=p Q=½ρv₀²·Q=½ρv₀³A₀ and the relation can be seen in FIG. 14.

In the above description of exemplary embodiments and examples, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A capsule device suitable for ingestion or insertion into a lumen of a human or animal subject, wherein the capsule device comprises:
a capsule housing,
a drug outlet arranged relative to the capsule housing,
a drug reservoir configured to accommodate a liquid drug substance,
an actuation chamber (A), and
a drug expelling unit, wherein the drug expelling unit is configured for being actuated to expel the liquid drug substance through the drug outlet, wherein the drug expelling unit comprises a gas expansion unit actuatable to generate pressurized gas in the actuation chamber (A), or release gas from the actuation chamber (A), for exerting load onto the liquid drug substance,
wherein the drug reservoir is provided as a single capillary duct having a first end and a second end, the single capillary duct configured for fluidically connecting the actuation chamber (A) with the drug outlet, wherein the liquid drug substance is arranged within the single capillary duct, and
wherein a gas release gate is arranged to control flow of pressurized gas from the actuation chamber (A) towards the drug outlet, wherein the gas release gate is configured for being operated between:
a) a first configuration wherein pressurized gas in the actuation chamber (A) is prevented from forcing liquid drug substance from the single capillary duct through the drug outlet, and
b) a second configuration wherein pressurized gas from the actuation chamber (A) is permitted to force liquid drug substance from the single capillary duct through the drug outlet.

2. The capsule device as defined in claim 1, wherein in the second configuration pressurized gas from the actuation chamber (A) engages directly with the liquid drug substance in the single capillary duct thereby exerting load onto the liquid drug substance to force the liquid drug substance towards the drug outlet.

3. The capsule device as defined in claim 1, wherein the liquid drug substance in the single capillary duct arranged closest to the actuation chamber (A) defines a liquid-gas interface.

4. The capsule device as defined in claim 1, wherein in the second configuration pressurized gas from the actuation chamber (A) engages directly with the liquid drug substance in the single capillary duct to exert load onto the liquid drug substance.

5. The capsule device as defined in claim 1, wherein the single capillary duct between the first end and the second end forms an elongated capillary forming a non-rectilinear configuration, such as a coiled configuration.

6. The capsule device as defined in claim 1, wherein the capsule device is sized and configured for being ingested into a gastrointestinal lumen of a human.

7. The capsule device as defined in claim 1, wherein the lumen comprises a lumen wall, wherein the drug outlet comprises a nozzle arrangement configured for needleless delivery, and wherein the capsule is configured to expel the liquid drug substance through the nozzle arrangement with a penetration velocity allowing the liquid drug substance to penetrate tissue of the lumen wall.

8. The capsule device as defined in claim 1, wherein the lumen comprises a lumen wall, wherein the drug outlet comprises an injection needle configured to deliver the liquid drug substance from the single capillary duct through a lumen of the injection needle.

9. The capsule device as defined in claim 1, wherein the gas expansion unit comprises a gas generator configured actuatable to generate pressurized gas in the actuation chamber (A) for exerting load on the liquid drug substance, and
wherein a burst gate is arranged between the gas generator and the single capillary duct, the burst gate being configured to release load onto liquid drug substance in the single capillary duct upon increase in gas pressure in the actuation chamber (A) above a threshold pressure level to thereby initiate expelling of the liquid drug substance.

10. The capsule device as defined in claim 9, wherein the gas generator comprises a trigger arrangement configured to actuate the gas generator.

11. The capsule device as defined in claim 9, wherein the burst gate comprises a rupturable membrane, such as a burst disc.

12. The capsule device as defined in claim 1, wherein the gas expansion unit comprises a gas canister filled with pressurized gas and comprising a rupturable seal configured rupturable for allowing pressurized gas from the gas canister to flow to the actuation chamber (A) upon rupturing of the rupturable seal.

13. The capsule device as defined in claim 12, wherein the gas release gate is defined or comprises said rupturable seal.

14. The capsule device as defined in claim 12, wherein the capsule device comprises a trigger arrangement comprising a spike, wherein the spike and the pressurized gas canister are arranged for relative movement, and wherein the trigger arrangement comprises means structure for creating relative movement between the spike and the pressurized gas canister to rupture the rupturable seal.

15. The capsule device as defined in claim 1, wherein the cross-sectional area of the single capillary duct, at least along a part of its extension, is dimensioned between 1 mm² and 16 mm².
